(19) United States
(12) Patent Application Publication
(10) Pub. No.: US 2026/0184716 A1
(43) Pub. Date: Jul. 2, 2026

(54) SOLID FORMS OF A FUSED PYRIDINE FOR THE TREAMTENT OF CANCER

(71) Applicant: Novartis AG, Basel (CH)

(72) Inventors: Jvan BRUN, Muttenz (CH); Jacques HAMON, Habsheim (FR); Juergen Hans-Hermann HINRICHS, Schopfheim (DE); Ziyue HONG, Shanghai (CN); Henrik MOEBITZ, Arlesheim (CH); Anett PERLBERG, Wolfwil (CH); Joseph SCHOEPFER, Ependes (FR); Ross Sinclair STRANG, Hagenthal-le-Bas (FR); Chang WANG, Zhenjiang City (CN)

(21) Appl. No.: 19/130,582

(22) PCT Filed: Nov. 21, 2023
(86) PCT No.: PCT/IB2023/061715

§ 371 Date: May 16, 2025

(30) Foreign Application Priority Data
Nov. 23, 2022 (WO) PCT/CN2022/133692

(51) Int. Cl. C07D 487/04; A61K 31/519

(52) U.S. Cl. CPC C07D 487/04; A61K 31/519

(57) ABSTRACT

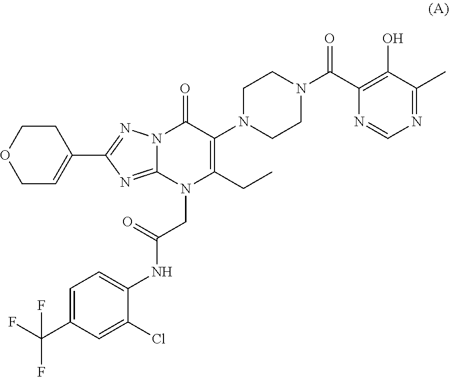 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

The present invention relates to solid forms of Compound (A), and a pharmaceutical composition comprising said solid form. The solid form of Compound (A) of the present invention, or the pharmaceutical composition of the present invention, can be used as a medicament.

### FIELD OF THE INVENTION

The present invention relates to solid forms of N-(2-chloro-4-(trifluoromethyl)phenyl)-2-(2-(3,6-dihydro-2H-pyran-4-yl)-5-ethyl-6-(4-(5-hydroxy-6-methylpyrimidine-4-carbonyl) piperazin-1-yl)-7-oxo-[1,2,4]triazolo[1,5-a]pyrimidin-4 (7H)-yl) acetamide, hereinafter referred to as ‘Compound A’. Compound A can be used as a medicament, in particular for the treatment of a disease such as cancer, in particular cancer that is treated by WRN inhibition, Compound A being an inhibitor of Werner Syndrome RecQ DNA Helicase (WRN). Said compound is in particular for use in the treatment of cancer that is characterized as microsatellite instability-high (MSI-H) or mismatch repair deficient (dMMR).

### BACKGROUND OF THE INVENTION

Loss of DNA mismatch repair is a common initiating event in cancer development occurring in 10-30% of colorectal, endometrial, ovarian and gastric cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). Cancers that have lost competence in mismatch repair (MMR) have a high mutational burden, and frequent deletion and insertion events in repetitive DNA tracts, a phenotype known as microsatellite instability (MSI). While progress has been made in the treatment of microsatellite instability high (MSI-H) cancers, and the demonstration that pembrolizumab (anti-PD1) treatment led to significantly longer progression-free survival than chemotherapy when received as first-line therapy for MSI-H-dMMR metastatic colorectal cancer resulted in the recent approval of pembrolizumab as first-line treatment of these cancers, there is still a significant unmet medical need in CRC and other MSI-H indications (André T., et al. Pembrolizumab in Microsatellite-Instability-High Advanced Colorectal Cancer. N Engl J Med 383 (23): 2207-2218 (2020)). Several large-scale functional genomics screens across large panels of cell lines, including Novartis with 398 cell lines from the Cancer Cell Line Encyclopedia (CCLE) (McDonald E. R. et al., Project DRIVE: A Compendium of Cancer Dependencies and Synthetic Lethal Relationships Uncovered by Large-Scale, Deep RNAi Screening. Cell 170 (3): 577-592 (2017)), have identified the Werner Syndrome RecQ helicase (WRN) as being selectively required for the survival of cell lines with defective mismatch repair that have become MSI-H (Behan, F. M. et al. Prioritization of cancer therapeutic targets using CRISPR-Cas9 screens. Nature 568, 511-516 (2019), Chan, E. M. et al. WRN helicase is a synthetic lethal target in microsatellite unstable cancers. Nature 568, 551-556 (2019). Kategaya, L., Perumal, S. K., Hager, J. H. & Belmont, L. D. Werner syndrome helicase is required for the survival of cancer cells with microsatellite instability. iScience 13, 488-497 (2019), Lieb, S. et al. Werner syndrome helicase is a selective vulnerability of microsatellite instability-high tumor cells. eLife 8, e43333 (2019)). WRN is synthetic lethal with MSI cancers. Depletion of WRN leads to anti-proliferative effects and results in activation of multiple DNA damage signaling markers, induction of cell cycle arrest and apoptosis in MMR cancer models but not cancer cells with an intact MMR pathway. These findings indicate that WRN provides a DNA repair and maintenance function that is essential for cell survival in MSI cancers. Recently, the mechanism of WRN dependence has been elucidated. It has been shown that dinucleotide TA repeats are selectively unstable in MSI cells and undergo large scale expansions. These expanded TA repeats form secondary DNA structures that require the WRN helicase for unwinding (van Wietmarschen, N. et al. Repeat expansions confer WRN dependence in microsatellite-unstable cancers. Nature 586, 292-298, 2020). In the absence of WRN (or upon WRN helicase inhibition), expanded TA repeats in MSI cells are subject to nuclease cleavage and chromosome breakage. Thus, inhibiting the WRN helicase is an attractive strategy for the treatment of mismatch repair defective cancers.

N-(2-chloro-4-(trifluoromethyl)phenyl)-2-(2-(3,6-dihydro-2H-pyran-4-yl)-5-ethyl-6-(4-(5-hydroxy-6-methylpyrimidine-4-carbonyl) piperazin-1-yl)-7-oxo-[1,2,4]triazolo[1,5-a]pyrimidin-4 (7H)-yl) acetamide, named herein as ‘Compound A’, is an inhibitor of Werner Syndrome RecQ DNA Helicase (WRN), and is disclosed in patent application PCT/IB2022/054850, published as WO2022/249060. The content of patent application PCT/IB2022/054850 is hereby incorporated by reference.

Compound A, or N-(2-chloro-4-(trifluoromethyl)phenyl)-2-(2-(3,6-dihydro-2H-pyran-4-yl)-5-ethyl-6-(4-(5-hydroxy-6-methylpyrimidine-4-carbonyl) piperazin-1-yl)-7-oxo-[1,2,4]triazolo[1,5-a]pyrimidin-4 (7H)-yl) acetamide has the structure:

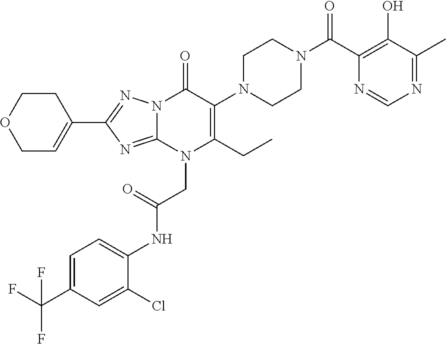 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

Methods for preparation of Compound A are disclosed in the synthetic procedures of patent application PCT/IB2022/054850. Compound A is disclosed specifically in Examples 42 and 123 of said patent application, and these Examples are hereby incorporated by reference. Compound A crystalline form ‘Modification A’, also designated as “Form A”, is disclosed in PCT/IB2022/054850. Additional new crystalline forms are disclosed herein. All forms herein are the free form (no salt) of Compound A.

Different solid state forms of an active pharmaceutical ingredient often possess different properties. Differences in physicochemical properties of solid forms can play a crucial role for the improvement of pharmaceutical compositions, for example, pharmaceutical formulations with improved dissolution profile or with improved stability or shelf-life can become accessible due to an improved solid state form of an active pharmaceutical ingredient. Also processing or handling of the active pharmaceutical ingredient during the formulation process may be improved. New solid state forms of an active pharmaceutical ingredient can thus have desirable processing properties. They can be easier to handle, better suited for storage, and/or allow for better purification, compared to previously known solid forms. The present invention therefore provides alternative solid state forms of Compound A which possess different physicochemical properties.

### SUMMARY OF THE INVENTION

In a first aspect of the invention, there is provided a crystalline form of Compound A (Modification B), wherein the crystalline form is characterized by a X-ray powder diffraction pattern comprising 4 or more reflections at 2-Theta angles selected from a group consisting of: 5.6±0.2°, 11.2±0.2°, 12.6±0.2°, 14.8±0.2°, 17.4±0.2°, 18.1±0.2°, 19.2±0.2°, 22.0±0.2°, 22.4±0.2°, 25.1±0.2°, 25.3±0.2°, 26.2±0.2°, 27.3±0.2°, 29.7±0.2° and 34.5±0.2°, when measured at a temperature in the range of from 20 to 30° C. with Cu-Kalpha radiation having a wavelength of 0.15418 nm.

In a second aspect of the invention, there is provided a crystalline form of Compound A (Modification C), wherein the crystalline form is characterized by a X-ray powder diffraction pattern comprising 4 or more reflections at 2-Theta angles selected from a group consisting of: 4.0±0.2°, 8.0±0.2°, 8.7±0.2°, 12.1±0.2°, 14.5±0.2°, 15.1±0.2°, 15.5±0.2°, 16.2±0.2°, 16.7±0.2°, 17.9±0.2°, 19.8±0.2°, 20.2±0.2°, 20.6±0.2°, 23.4±0.2°, 24.2±0.2°, 25.4±0.2°, 26.5±0.2°, 28.5±0.2°, 32.1±0.2°, when measured at a temperature in the range of from 20 to 30° C. with Cu-Kalpha radiation having a wavelength of 0.15418 nm.

In a third aspect of the invention, there is provided a crystalline form of Compound A (Modification D), wherein the crystalline form is characterized by a X-ray powder diffraction pattern comprising 4 or more reflections at 2-Theta angles selected from a group consisting of: 8.5±0.2°, 9.1±0.2°, 11.6±0.2°, 13.7±0.2°, 14.3±0.2°, 15.0±0.2°, 15.4±0.2°, 16.9±0.2°, 17.5±0.2°, 19.1±0.2°, 19.9±0.2°, 20.2±0.2°, 21.5±0.2°, 22.6±0.2°, 23.5±0.2°, 25.6±0.2°, 26.4±0.2°, 26.8±0.2°, 28.8±0.2°, 30.3±0.2°, when measured at a temperature in the range of from 20 to 30° C. with Cu-Kalpha radiation having a wavelength of 0.15418 nm.

In a fourth aspect of the invention, there is provided a crystalline form of Compound A (Hydrate H_{A}), wherein the crystalline form is characterized by a X-ray powder diffraction pattern comprising 4 or more reflections at 2-Theta angles selected from a group consisting of: 7.8±0.2°, 8.4±0.2°, 11.8±0.2°, 15.7±0.2°, 16.4±0.2°, 16.8±0.2°, 17.4±0.2°, 17.7±0.2°, 19.7±0.2°, 20.2±0.2°, 24.3±0.2°, 24.7±0.2°, 25.1±0.2°, 26.6±0.2°, 29.0±0.2°, when measured at a temperature in the range of from 20 to 30° C. with Cu-Kalpha radiation having a wavelength of 0.15418 nm.

In a fifth aspect of the invention, there is provided a crystalline form of Compound A (Hydrate H_{B}), wherein the crystalline form is characterized by a X-ray powder diffraction pattern comprising 4 or more reflections at 2-Theta angles selected from a group consisting of: 9.0±0.2°, 13.6±0.2°, 14.7±0.2°, 15.8±0.2°, 16.1±0.2°, 16.4±0.2°, 17.4±0.2°, 18.4±0.2°, 19.3±0.2°, 20.3±0.2°, 22.1±0.2°, 22.7±0.2°, 23.1±0.2°, 24.8±0.2°, 25.4±0.2°, 29.6±0.2°, 30.1±0.2°, when measured at a temperature in the range of from 20 to 30° C. with Cu-Kalpha radiation having a wavelength of 0.15418 nm.

In a sixth aspect of the invention, there is provided a crystalline form of Compound A (Hydrate H_{C}), wherein the crystalline form is characterized by a X-ray powder diffraction pattern comprising 4 or more reflections at 2-Theta angles selected from a group consisting of: 8.0±0.2°, 8.7±0.2°, 11.8±0.2°, 12.2±0.2°, 14.9±0.2°, 15.2±0.2°, 15.6±0.2°, 16.4±0.2°, 16.8±0.2°, 19.7±0.2°, 20.1±0.2°, 20.5±0.2°, 21.1±0.2°, 24.3±0.2°, 24.5±0.2°, 25.1±0.2°, 25.6±0.2°, 26.6±0.2°, 33.9±0.2°, when measured at a temperature in the range of from 20 to 30° C. with Cu-Kalpha radiation having a wavelength of 0.15418 nm.

In a seventh aspect of the invention, there is provided a crystalline form of Compound A (Solvate S_{A}), wherein the crystalline form is characterized by a X-ray powder diffraction pattern comprising 4 or more reflections at 2-Theta angles selected from a group consisting of: 7.8±0.2°, 11.8±0.2°, 15.3±0.2°, 15.7±0.2°, 16.8±0.2°, 19.6±0.2°, 20.8±0.2°, 21.9±0.2°, 23.6±0.2°, 24.3±0.2°, 27.6±0.2°, when measured at a temperature in the range of from 20 to 30° C. with Cu-Kalpha radiation having a wavelength of 0.15418 nm.

In an eighth aspect of the invention, there is provided a crystalline form of Compound A (Solvate S_{B}), wherein the crystalline form is characterized by a X-ray powder diffraction pattern comprising 4 or more reflections at 2-Theta angles selected from a group consisting of: 9.5±0.2°, 11.3±0.2°, 14.4±0.2°, 14.8±0.2°, 15.2±0.2°, 16.9±0.2°, 17.1±0.2°, 17.7±0.2°, 18.0±0.2°, 18.5±0.2°, 18.7±0.2°, 19.1±0.2°, 19.6±0.2°, 20.3±0.2°, 20.9±0.2°, 22.1±0.2°, 23.4±0.2°, 27.1±0.2°, 28.4±0.2°, 28.8±0.2°, when measured at a temperature in the range of from 20 to 30° C. with Cu-Kalpha radiation having a wavelength of 0.15418 nm.

In a ninth aspect of the invention, there is provided a method of treating and/or preventing cancer, in particular cancer that is characterized as microsatellite instability-high (MSI-H) or mismatch repair deficient (dMMR), said method comprising administering an effective amount of the solid form according to any of the first to eighth aspects.

In a tenth aspect of the invention, there is provided a pharmaceutical composition comprising a crystalline form according to any of the first to eighth aspects, and at least one pharmaceutically acceptable excipient.

### BRIEF DESCRIPTION OF THE FIGURES

FIG. 1 shows the XRPD pattern of Compound A Modification A

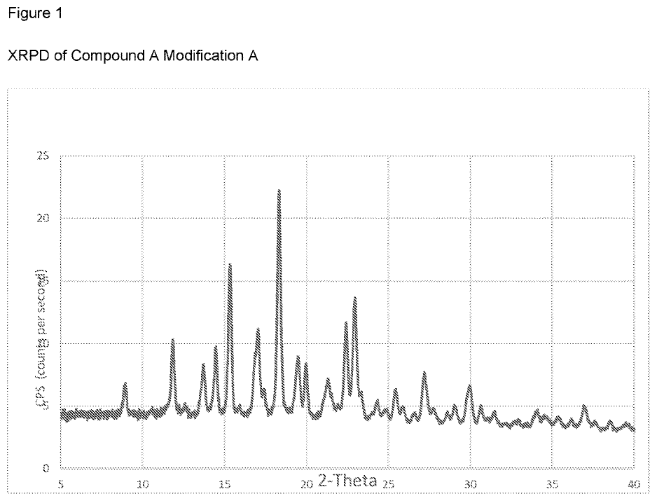

FIG. 2 shows the XRPD pattern of compound A Modification B

FIG. 3 shows the XRPD pattern of compound A Modification C

FIG. 4 shows the XRPD pattern of compound A Modification D

FIG. 5 shows the XRPD pattern of compound A Hydrate H_{A}

FIG. 6 shows the XRPD pattern of compound A Hydrate H_{B}

FIG. 7 shows the XRPD pattern of compound A Hydrate H_{C}

FIG. 8 shows the XRPD pattern of compound A Solvate S_{A}

FIG. 9 shows the XRPD pattern of compound A Solvate S_{B}

FIG. 10 shows the XRPD pattern of compound A Hydrate H_{A}

FIG. 11 shows the XRPD pattern of compound A amorphous form

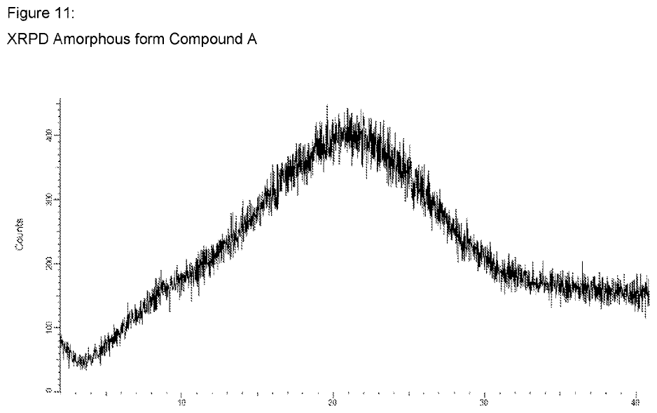

FIG. 12 shows the XRPD pattern of Compound A Modification A using a different instrument method compared to FIG. 1.

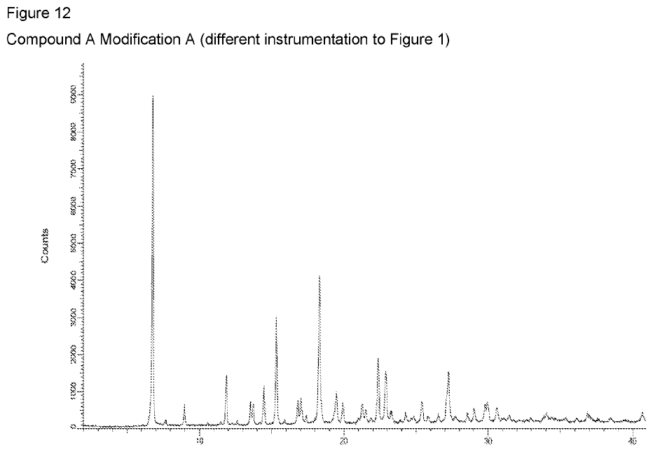

### DETAILED DESCRIPTION OF THE INVENTION

In one embodiment, there is provided a crystalline form of Compound A (Modification B), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 5.6±0.2°, 11.2±0.2°, 12.6±0.2°, 14.8±0.2°, 17.4±0.2°, 18.1±0.2°, 19.2±0.2°, 22.0±0.2°, 22.4±0.2°, 25.1±0.2°, 25.3±0.2°, 26.2±0.2°, 27.3±0.2°, 29.7±0.2° and 34.5±0.2°, when measured at a temperature in the range of from 20 to 30° C. with Cu-Kalpha radiation having a wavelength of 0.15418 nm.

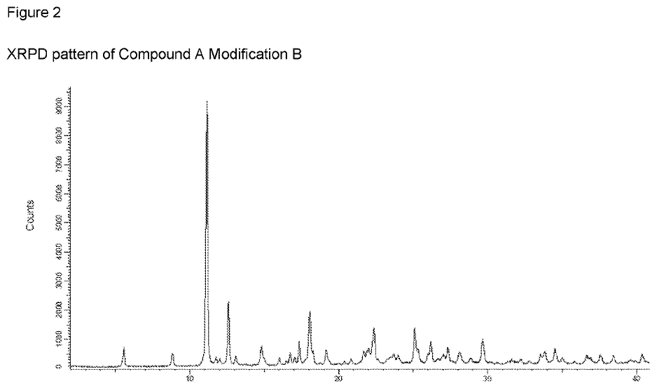

In another embodiment, there is provided a crystalline form of Compound A (Modification B), wherein said crystalline form is characterized by a X-ray diffraction pattern substantially the same as the X-ray powder diffraction pattern shown in FIG. 2, when measured at a temperature in the range of from 20 to 30° C. with Cu-Kalpha radiation having a wavelength of 0.15418 nm.

In another embodiment, there is provided a crystalline form of Compound A (Modification C), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 4.0±0.2°, 8.0±0.2°, 8.7±0.2°, 12.1±0.2°, 14.5±0.2°, 15.1±0.2°, 15.5±0.2°, 16.2±0.2°, 16.7±0.2°, 17.9±0.2°, 19.8±0.2°, 20.2±0.2°, 20.6±0.2°, 23.4±0.2°, 24.2±0.2°, 25.4±0.2°, 26.5±0.2°, 28.5±0.2°, 32.1±0.2°, when measured at a temperature in the range of from 20 to 30° C. with Cu-Kalpha radiation having a wavelength of 0.15418 nm.

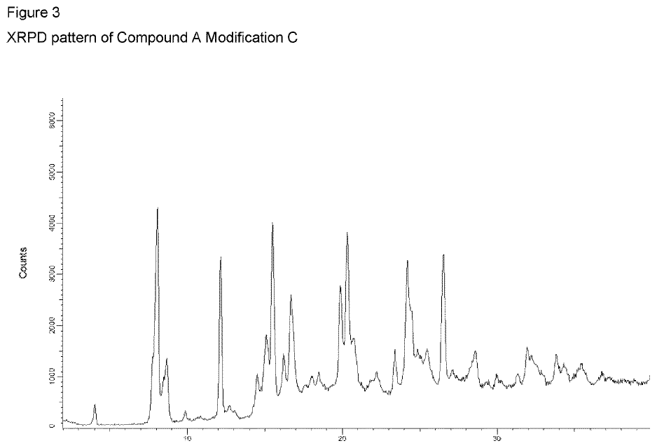

In another embodiment, there is provided a crystalline form of Compound A (Modification C), wherein said crystalline form is characterized by a X-ray diffraction pattern substantially the same as the X-ray powder diffraction pattern shown in FIG. 3, when measured at a temperature in the range of from 20 to 30° C. with Cu-Kalpha radiation having a wavelength of 0.15418 nm.

In another embodiment, there is provided a crystalline form of Compound A (Modification D), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 8.5±0.2°, 9.1±0.2°, 11.6±0.2°, 13.7±0.2°, 14.3±0.2°, 15.0±0.2°, 15.4±0.2°, 16.9±0.2°, 17.5±0.2°, 19.1±0.2°, 19.9±0.2°, 20.2±0.2°, 21.5±0.2°, 22.6±0.2°, 23.5±0.2°, 25.6±0.2°, 26.4±0.2°, 26.8±0.2°, 28.8±0.2°, 30.3±0.2°, when measured at a temperature in the range of from 20 to 30° C. with Cu-Kalpha radiation having a wavelength of 0.15418 nm.

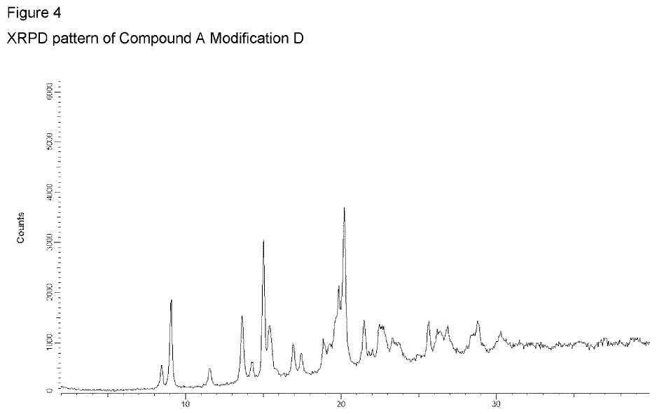

In another embodiment, there is provided a crystalline form of Compound A (Modification D), wherein said crystalline form is characterized by a X-ray diffraction pattern substantially the same as the X-ray powder diffraction pattern shown in FIG. 4, when measured at a temperature in the range of from 20 to 30° C. with Cu-Kalpha radiation having a wavelength of 0.15418 nm.

In another embodiment, there is provided a crystalline form of Compound A (Hydrate H_{A}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 7.8±0.2°, 8.4±0.2°, 11.8±0.2°, 15.7±0.2°, 16.4±0.2°, 16.8±0.2°, 17.4±0.2°, 17.7±0.2°, 19.7±0.2°, 20.2±0.2°, 24.3±0.2°, 24.7±0.2°, 25.1±0.2°, 26.6±0.2°, 29.0±0.2°, when measured at a temperature in the range of from 20 to 30° C. with Cu-Kalpha radiation having a wavelength of 0.15418 nm.

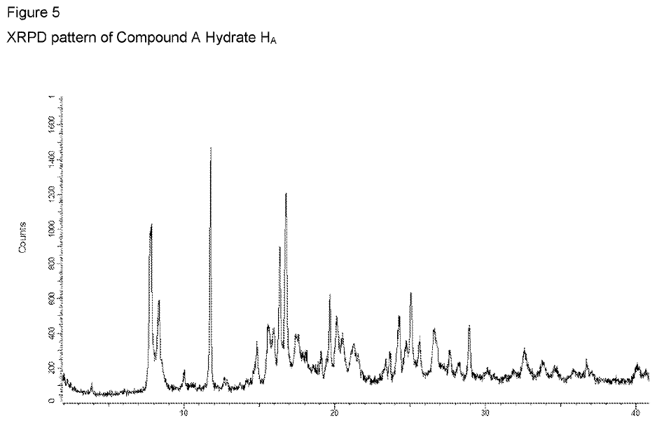

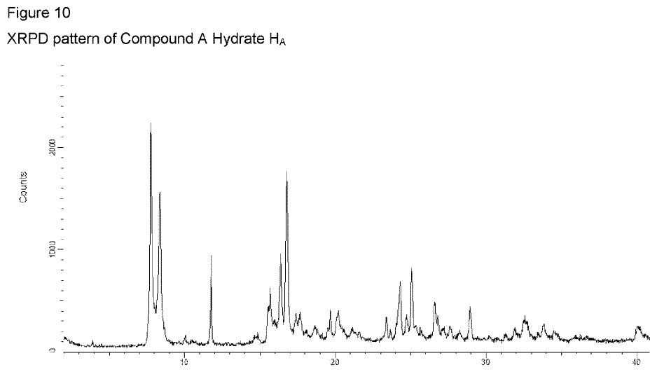

In another embodiment, there is provided a crystalline form of Compound A (Hydrate H_{A}), wherein said crystalline form is characterized by a X-ray diffraction pattern substantially the same as the X-ray powder diffraction pattern shown in FIG. 5 or FIG. 10, when measured at a temperature in the range of from 20 to 30° C. with Cu-Kalpha radiation having a wavelength of 0.15418 nm.

In another embodiment, there is provided a crystalline form of Compound A (Hydrate H_{B}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 9.0±0.2°, 13.6±0.2°, 14.7±0.2°, 15.8±0.2°, 16.1±0.2°, 16.4±0.2°, 17.4±0.2°, 18.4±0.2°, 19.3±0.2°, 20.3±0.2°, 22.1±0.2°, 22.7±0.2°, 23.1±0.2°, 24.8±0.2°, 25.4±0.2°, 29.6±0.2°, 30.1±0.2°, when measured at a temperature in the range of from 20 to 30° C. with Cu-Kalpha radiation having a wavelength of 0.15418 nm.

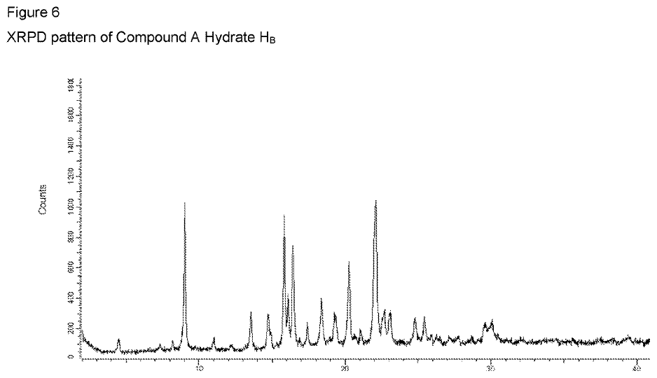

In another embodiment, there is provided a crystalline form of Compound A (Hydrate H_{B}), wherein said crystalline form is characterized by a X-ray diffraction pattern substantially the same as the X-ray powder diffraction pattern shown in FIG. 6, when measured at a temperature in the range of from 20 to 30° C. with Cu-Kalpha radiation having a wavelength of 0.15418 nm.

In another embodiment, there is provided a crystalline form of Compound A (Hydrate H_{C}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 8.0±0.2°, 8.7±0.2°, 11.8±0.2°, 12.2±0.2°, 14.9±0.2°, 15.2±0.2°, 15.6±0.2°, 16.4±0.2°, 16.8±0.2°, 19.7±0.2°, 20.1±0.2°, 20.5±0.2°, 21.1±0.2°, 24.3±0.2°, 24.5±0.2°, 25.1±0.2°, 25.6±0.2°, 26.6±0.2°, 33.9±0.2°, when measured at a temperature in the range of from 20 to 30° C. with Cu-Kalpha radiation having a wavelength of 0.15418 nm.

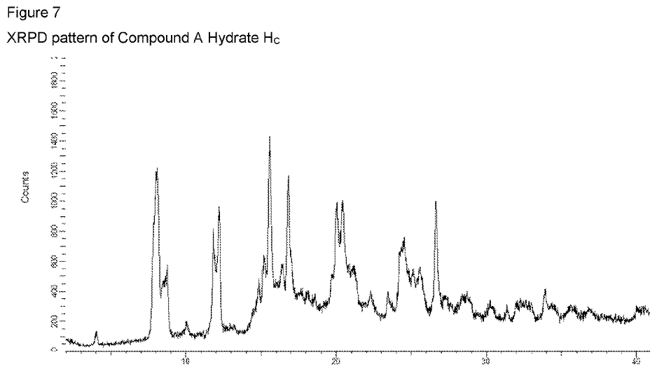

In another embodiment, there is provided a crystalline form of Compound A (Hydrate H_{C}), wherein said crystalline form is characterized by a X-ray diffraction pattern substantially the same as the X-ray powder diffraction pattern shown in FIG. 7, when measured at a temperature in the range of from 20 to 30° C. with Cu-Kalpha radiation having a wavelength of 0.15418 nm.

In another embodiment, there is provided a crystalline form of Compound A (Solvate S_{A}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 7.8±0.2°, 11.8±0.2°, 15.3±0.2°, 15.7±0.2°, 16.8±0.2°, 19.6±0.2°, 20.8±0.2°, 21.9±0.2°, 23.6±0.2°, 24.3±0.2°, 27.6±0.2°, when measured at a temperature in the range of from 20 to 30° C. with Cu-Kalpha radiation having a wavelength of 0.15418 nm.

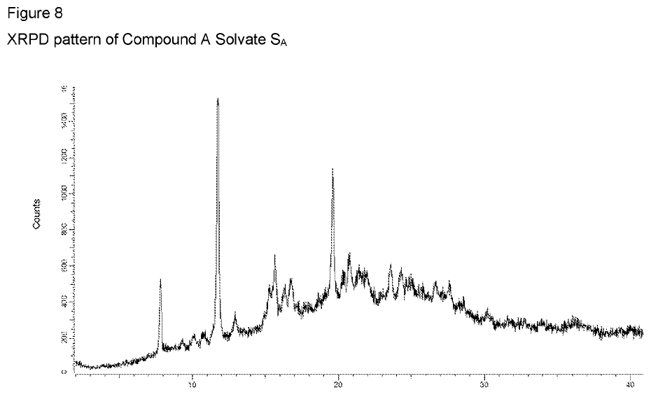

In another embodiment, there is provided a crystalline form of Compound A (Solvate S_{A}), wherein said crystalline form is characterized by a X-ray diffraction pattern substantially the same as the X-ray powder diffraction pattern shown in FIG. 8, when measured at a temperature in the range of from 20 to 30° C. with Cu-Kalpha radiation having a wavelength of 0.15418 nm.

In another embodiment, there is provided a crystalline form of Compound A (Solvate S_{B}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 9.5±0.2°, 11.3±0.2°, 14.4±0.2°, 14.8±0.2°, 15.2±0.2°, 16.9±0.2°, 17.1±0.2°, 17.7±0.2°, 18.0±0.2°, 18.5±0.2°, 18.7±0.2°, 19.1±0.2°, 19.6±0.2°, 20.3±0.2°, 20.9±0.2°, 22.1±0.2°, 23.4±0.2°, 27.1±0.2°, 28.4±0.2°, 28.8±0.2°, when measured at a temperature in the range of from 20 to 30° C. with Cu-Kalpha radiation having a wavelength of 0.15418 nm.

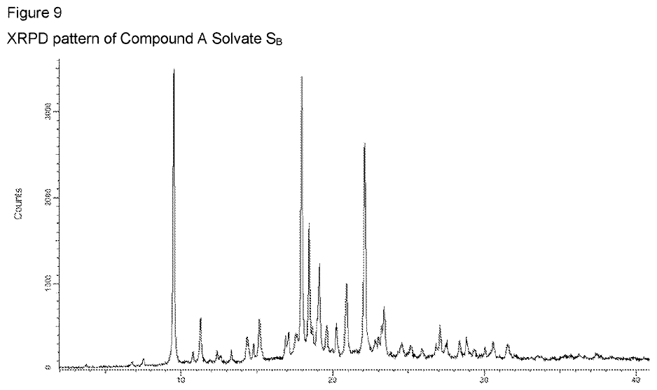

In another embodiment, there is provided a crystalline form of Compound A (Solvate S_{B}), wherein said crystalline form is characterized by a X-ray diffraction pattern substantially the same as the X-ray powder diffraction pattern shown in FIG. 9, when measured at a temperature in the range of from 20 to 30° C. with Cu-Kalpha radiation having a wavelength of 0.15418 nm.

### Composition

In another embodiment, the invention relates to a composition comprising at least 90 w-%, including at least 90, 91, 92, 93, 94, 95, 96, 97, 98 and 99 w-%, and also including equal to about 100 w-% of a crystalline form of Compound A as defined in any one of the embodiments herein, based on the total weight of the composition. The remaining material may comprise other solid form(s) of Compound A, and/or reaction impurities and/or processing impurities arising from the preparation of the composition.

### Pharmaceutical Compositions and Use

In a further aspect the present invention relates to the use of:
a solid form, such as crystalline form or amorphous form of Compound A of the present invention, or
the composition comprising an amorphous or crystalline form of Compound A of the present invention
   as defined in any one of the embodiments herein, for the preparation of a pharmaceutical composition.

In another aspect, the present invention relates to a pharmaceutical composition comprising a solid form of Compound A as defined in any of the embodiments herein and at least one pharmaceutically acceptable excipient.

The at least one pharmaceutically acceptable excipient, which is comprised in the pharmaceutical composition of the present invention, is preferably selected from the group consisting of fillers, diluents, binders, disintegrants, lubricants, glidants, and combinations thereof.

In a preferred embodiment, the pharmaceutical composition is an oral solid dosage form. Preferably the oral solid dosage form is selected from the group consisting of tablets, capsules, etc. In a particular preferred embodiment, the oral dosage form is a tablet or a capsule, most preferably a tablet.

In a further aspect, the present invention relates to a solid form of Compound A or the composition comprising the solid form of Compound A as defined in any one of the embodiments herein for use as a medicament.

In yet another aspect, the present invention relates to a solid form of Compound A or the composition comprising a solid form of Compound A as defined in any of the embodiments herein, for use in the treatment of cancer, in particular cancer that is characterized as microsatellite instability-high (MSI-H) or mismatch repair deficient (dMMR).

In another preferred embodiment, the invention concerns a method of treating and/or preventing cancer, in particular cancer that is characterized as microsatellite instability-high (MSI-H) or mismatch repair deficient (dMMR), said method comprising administering an effective amount of the solid form as defined in any of the embodiments herein, to a patient in need of such a treatment.

Crystalline forms disclosed herein

| | | |
|---|---|---|
| A | Modification A | Starting material, equilibration of Modification |
| | | B in solvents |
| B | Modification B | Starting material |
| C | Modification C | Prepared by drying of Hydrate H_{A} |
| D | Modification D | Prepared by drying of Hydrate H_{B} |
| H_{A} | Hydrate | Equilibration of Modification B in methanol, |
| | | dioxane and methanol/water mixture |
| H_{B} | Hydrate | Antisolvent precipitation from THF/water (90:10) |
| | | into water |
| H_{C} | Hydrate | From Modification C by water sorption |
| S_{A} | Solvate | Crystallization from a dichloromethane solution |
| S_{B} | Solvate | Crystallization from THF/IPAc |

Nine crystalline forms of Compound A are dislosed herein. Four anhydrous forms (Modification A, Modification B, Modification C and Modification D), three hydrates (Hydrate H_{A}, Hydrate H_{B} and Hydrate H_{C}) and two solvates (Solvate S_{A} and Solvate S_{B}). Figures illustrating the XRPD characterization data of all crystalline forms are included herein.

Modification B converted into Modification A through suspension equilibration in most solvents at 25° C. and 50° C. and in competitive suspension equilibration experiments with Modification A at 4° C. In methanol, dioxane and methanol/water mixtures, (partial) conversion to Hydrate H_{A} was observed. The formation of a hydrate from an anhydrous form requires the presence of water. Therefore, it is assumed that the methanol and dioxane used in the respective experiments still contained traces of water.

Hydrate H_{B} was obtained through antisolvent precipitation from THF/water into water.

Modification C was generated from Hydrate H_{A} only if the relative humidity was lower than % RH, and transformed into Hydrate H_{C} with moisture uptake.

Modification D was only obtained by heating Hydrate H_{B} above ˜84° C. during DSC analysis.

Solvate S_{A} was crystallized by evaporating a saturated solution of the drug substance in dichloromethane.

Solvate S_{B} was found during crystallization process development after cooling crystallization from THF/IPAC.

### Specific Examples and XRPD Data

TABLE 1
Peak list of strongest peaks of Modification B (rel. intensity ≥5%)

| Reflection position | Relative intensity |
|---|---|
| [°2-Theta] | [%] |
| 5.6 | 7.0 |
| 11.2 | 100.0 |
| 12.6 | 23.3 |
| 14.8 | 7.3 |
| 17.4 | 8.8 |
| 18.1 | 19.3 |
| 19.2 | 5.7 |
| 22.0 | 5.6 |
| 22.4 | 13.3 |
| 25.1 | 13.3 |
| 25.3 | 5.3 |
| 26.2 | 7.8 |
| 27.3 | 5.6 |
| 29.7 | 8.8 |
| 34.5 | 5.5 |

### Preparation of Modification B:

3.85 g Compound A was dissolved in 300 ml EtOH and 250 ml DCM at 40° C. and the solution concentrated under reduced pressure to 150 ml. After standing for 2 days at room temperature, the resulting suspension was sonicated for 1.5 h in a ultrasonic bath, then stirred for 2 hours at 0° C. and the crystalline material filtered, then washed with cold EtOH.

Modification B of Compound A (free form, no salt) is a crystalline powder consisting of plate-like particles. The material was of high purity and showed a melting point of about 239° C. with concurrent recrystallization and subsequent second melting at about 274° C.

TABLE 2
Peak list of strongest peaks of Modification C

| Reflection position | Relative intensity |
|---|---|
| [°2-Theta] | [%] |
| 4.0 | 10.6 |
| 8.0 | 100.0 |
| 8.7 | 29.8 |
| 12.1 | 75.0 |
| 14.5 | 13.7 |
| 15.1 | 30.6 |
| 15.5 | 88.3 |
| 16.2 | 20.4 |
| 16.7 | 41.6 |
| 17.9 | 10.3 |
| 19.8 | 43.0 |
| 20.2 | 73.8 |
| 20.6 | 20.2 |
| 23.4 | 14.8 |
| 24.2 | 36.1 |
| 25.4 | 15.7 |
| 26.5 | 48.4 |
| 28.5 | 16.1 |
| 32.1 | 14.0 |

### Preparation of Modification C:

About 50 mg of Compound A (crystalline form: Hydrate H_{A}) was exposed to dry nitrogen (0% rel. humidity) at room temperature for about 3 hours. Full conversion to Modification C is observed.

TABLE 3
Peak list of strongest peaks of Modification D

| Reflection position | Relative intensity |
|---|---|
| [°2-Theta] | [%] |
| 8.5 | 14.2 |
| 9.1 | 53.8 |
| 11.6 | 10.7 |
| 13.7 | 40.4 |
| 14.3 | 10.8 |
| 15.0 | 81.8 |
| 15.4 | 32.3 |
| 16.9 | 18.7 |
| 17.5 | 13.0 |
| 19.1 | 11.6 |
| 19.9 | 50.8 |
| 20.2 | 100.0 |
| 21.5 | 26.8 |
| 22.6 | 22.1 |
| 23.5 | 11.0 |
| 25.6 | 21.4 |
| 26.4 | 14.7 |
| 26.8 | 17.0 |
| 28.8 | 18.2 |
| 30.3 | 11.4 |

### Preparation of Modification D:

About 50 mg of Compound A (crystalline form: Hydrate H_{B}) was heated to 100° C. using a heating rate of 20 K/min or less. Full conversion to Modification D is observed.

TABLE 4
Peak list of strongest peaks of Hydrate H_{A}

| Reflection position | Relative intensity |
|---|---|
| [°2-Theta] | [%] |
| 7.8 | 100.0 |
| 8.4 | 68.1 |
| 11.8 | 40.0 |
| 15.7 | 23.7 |
| 16.4 | 38.6 |
| 16.8 | 74.4 |
| 17.4 | 10.6 |
| 17.7 | 11.8 |
| 19.7 | 12.7 |
| 20.2 | 11.5 |
| 24.3 | 25.4 |
| 24.7 | 10.3 |
| 25.1 | 30.5 |
| 26.6 | 14.8 |
| 29.0 | 14.0 |

### Preparation of Hydrate H_{A}:

Example 1.H_{A}: About 50 mg of Compound A (Modification B) was mixed with 1 mL of solvent mixture methanol/water 97:3 v/v. The mixture was stirred at 800 rpm and 25° C. for 4 weeks. Additional solid drug substance might have to be added after a few days to ensure a suspension. The resulting suspension after 4 weeks was centrifuged at 13000 rpm for 3 min. Solid residue was analyzed with XRPD and conversion to Hydrate H_{A} was observed.

Example 2.H_{A}: The same experiment was performed using the solvent mixture methanol/water 90:10 v/v. This experiment also resulted in the formation of Hydrate H_{A}.

Example 3.H_{A}: About 200 mg Modification B was slurried in 4 mL of methanol overnight. The solid was separated by centrifugation and dried in air. Full conversion to Hydrate H_{A} was observed.

TABLE 5
Peak list of strongest peaks of Hydrate H_{B}

| Reflection position | Relative intensity |
|---|---|
| [°2-Theta] | [%] |
| 9.0 | 98.6 |
| 13.6 | 26.5 |
| 14.7 | 23.7 |
| 15.8 | 90.9 |
| 16.1 | 36.5 |
| 16.4 | 70.8 |
| 17.4 | 15.9 |
| 18.4 | 29.7 |
| 19.3 | 19.1 |
| 20.3 | 57.0 |
| 22.1 | 100.0 |
| 22.7 | 19.4 |
| 23.1 | 19.2 |
| 24.8 | 18.1 |
| 25.4 | 18.9 |
| 29.6 | 13.9 |
| 30.1 | 15.6 |

### Preparation of Hydrate H_{B}:

About 100 g of Compound A Modification B was dissolved completely in 1 mL of THF/water (90/10) by sonication. About 1 mL water was added dropwise as anti-solvent until precipitation occurred. The suspension was stirred overnight, the solid was separated by centrifugation and dried in air. Full conversion to Hydrate H_{B} was observed.

TABLE 6
Peak list of strongest peaks of Hydrate H_{C}

| Reflection position | Relative intensity |
|---|---|
| [°2-Theta] | [%] |
| 8.0 | 90.0 |
| 8.7 | 32.5 |
| 11.8 | 57.9 |
| 12.2 | 69.3 |
| 14.9 | 25.4 |
| 15.2 | 36.0 |
| 15.6 | 100.0 |
| 16.4 | 29.5 |
| 16.8 | 75.6 |
| 19.7 | 18.8 |
| 20.1 | 59.5 |
| 20.5 | 60.0 |
| 21.1 | 23.5 |
| 24.3 | 34.3 |
| 24.5 | 35.1 |
| 25.1 | 21.2 |
| 25.6 | 24.2 |
| 26.6 | 59.7 |
| 33.9 | 15.7 |

### Preparation of Hydrate H_{C}:

About 50 mg of Compound A (Hydrate H_{A}), was exposed to dry nitrogen for 3 hours followed by a further exposure to 40% rel. humidity for 72 hours (all at room temperature). Full conversion to Hydrate H_{C} was observed after this procedure

TABLE 7
Peak list of strongest peaks of Solvate S_{A}

| Reflection position | Relative intensity |
|---|---|
| [°2-Theta] | [%] |
| 7.8 | 30.8 |
| 11.8 | 100.0 |
| 15.3 | 13.7 |
| 15.7 | 28.3 |
| 16.8 | 14.9 |
| 19.6 | 53.2 |
| 20.8 | 18.7 |
| 21.9 | 12.3 |
| 23.6 | 15.0 |
| 24.3 | 12.9 |
| 27.6 | 10.8 |

### Preparation of Solvate S_{A}

A saturated solution of Compound A Modification B in dichloromethane was prepared at room temperature. The solution was filtered to give a clear solution and was left to evaporate slowly at room temperature. Crystallization of Solvate S_{A} was observed.

TABLE 8
Peak list of strongest peaks of Solvate S_{B}

| Reflection position | Relative intensity |
|---|---|
| [°2-Theta] | [%] |
| 9.5 | 100.0 |
| 11.3 | 15.3 |
| 14.4 | 8.2 |
| 14.8 | 5.5 |
| 15.2 | 13.0 |
| 16.9 | 7.1 |
| 17.1 | 8.2 |
| 17.7 | 7.0 |
| 18.0 | 94.9 |
| 18.5 | 44.1 |
| 18.7 | 9.2 |
| 19.1 | 30.4 |
| 19.6 | 9.9 |
| 20.3 | 9.7 |
| 20.9 | 24.2 |
| 22.1 | 71.9 |
| 23.4 | 16.1 |
| 27.1 | 10.6 |
| 28.4 | 5.5 |
| 28.8 | 6.5 |

### Preparation of Solvate S_{B}

2 g of Compound A Modification B was added to 40 mL tetrahydrofuran at 50° C. and the mixture was stirred for 30 min. The resulting solution was cooled to 35° C. in 10 min and was kept at this temperature for 30 min. 10 mg of Modification B were added followed by slow addition of 40 mL of isopropyl acetate within 8 hours. The resulting mixture was cooled to 0° C. and was stirred overnight. A suspension was obtained and was filtered and the filtercake was dried for two days. The obtained solid was found to represent a new crystalline form, termed Solvate S_{B}. Yield: 84%.

### Amorphous Form of Compound A

Amorphous form of Compound A is also disclosed herein. Amorphous form was prepared as follows. About 2 g of Compound A Modification B was completely dissolved in 40 mL of THF by sonication. The clear solution was added directly into an excess of heptane (1:10 v/v). The solid formed was separated immediately by centrifugation and dried under vacuum at 80° C. to remove residual solvent. Crystallinity was checked by XRPD, residual solvents by NMR and T_{g} by mDSC.

### XRPD Method (Modifications B, C, D and all Hydrates, Solvates and Amorphous Form)

| | |
|---|---|
| Instrument | Bruker D8 Advance |
| Detector | LynxEye (1D mode), open angle: 2.948° |
| Radiation | CuKα (0.15418 nm) |
| Monochromator | Ni-filter |
| X-ray generator power | 40 kV, 40 mA |
| Step size | 0.0164° or 0.0410° (2theta) |
| Time per step | 0.3 s |
| Scan range | 2°-40° 2theta |
| Scan time | 768 s or 279 s |
| Slits | Primary fixed illuminated sample size: 10 mm, |
| | secondary: open angle: 2.2°, axial soller: 2.5° |

### XRPD Methods (Modification A)

The instrumentation methods and synthetic methods for Modification A are described in WO2022/249060.

### Definitions

In the context of the present invention the following definitions have the indicated meaning, unless explicitly stated otherwise:

As used herein the term “room temperature” refers to a temperature in the range of from 20 to 30° C.

The term “reflection” with regard to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material, whereas amorphous material is defined as solid material, which lacks long-range order and only displays short-range order, thus resulting in broad scattering. According to literature, long-range order e.g. extends over approximately 100 to 1000 atoms, whereas short-range order is over a few atoms only (see “Fundamentals of Powder Diffraction and Structural Characterization of Materials” by Vitalij K. Pecharsky and Peter Y. Zavalij, Kluwer Academic Publishers, 2003, page 3).

The term “essentially the same” or “substantially the same” with reference to powder X-ray diffraction means that variabilities in reflection positions and relative intensities of the reflections are to be taken into account. For example, a typical precision of the 2-Theta values is in the range of ±0.2° 2-Theta, preferably in the range of ±0.1° 2-Theta. Furthermore, one skilled in the art will appreciate that relative reflection intensities will show inter-apparatus variability as well as variability due to degree of crystallinity, preferred orientation, particle size, sample preparation and other factors known to those skilled in the art and should be taken as qualitative measure only.

Crystalline forms of Compound A of the present invention may be referred to herein as being characterized by graphical data “as shown in” a figure. Such data include, for example, powder X-ray diffraction. The person skilled in the art understands that factors such as variations in instrument type, response and variations in sample directionality, sample concentration and sample purity may lead to small variations for such data when presented in graphical form, for example variations relating to the exact peak positions and intensities. However, a comparison of the graphical data in the figures herein with the graphical data generated for another or an unknown solid form and the confirmation that two sets of graphical data relate to the same crystal form is well within the knowledge of a person skilled in the art.

The terms “solid form” or “solid state form” as used herein refer to any crystalline and/or amorphous phase of a compound.

As used herein, the term “amorphous” refers to a solid form of a compound that is not crystalline. An amorphous compound possesses no long-range order and does not display a definitive X-ray diffraction pattern with reflections.

‘X-ray powder diffraction pattern’ means an X-ray powder diffractogram.

As used herein the term “Modification” is used to refer to different crystalline forms having the same chemical composition but different spatial arrangements of the molecules, atoms, and/or ions forming the crystal.

The term “hydrate” as used herein, refers to a crystalline solid where either water is cooperated in or accommodated by the crystal structure e.g. is part of the crystal structure or entrapped into the crystal (water inclusions). Thereby, water can be present in a stoichiometric or non-stoichiometric amount. When water is present in stoichiometric amount, the hydrate may be referred to by adding greek numeral prefixes. For example, a hydrate may be referred to as a hemihydrate or as a monohydrate depending on the water/compound stoichiometry. The water content can be measured, for example, by Karl-Fischer-Coulometry.

As used herein “solvate” refers to a crystalline form of a molecule, atom, and/or ions that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. Solvates may occur as dimers or oligomers comprising more than one molecule or Compound A within the crystalline lattice structure.

The terms “anhydrous form” or “anhydrate” as used herein refer to a crystalline solid where no water is cooperated in or accommodated by the crystal structure. Anhydrous forms may still contain residual water, which is not part of the crystal structure but may be adsorbed on the surface or absorbed in disordered regions of the crystal. Typically, an anhydrous form does not contain more than 2.0 w-%, preferably not more than 1.0 w-% of water, based on the weight of the crystalline form.

The term “effective amount” as used herein with regard to Compound A encompasses an amount of Compound A, which causes the desired therapeutic and/or prophylactic effect.

As used herein, the term “about” means within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

As used herein, “substantially pure,” when used in reference to a form, means a compound having a purity greater than 90 w-%, including greater than 90, 91, 92, 93, 94, 95, 96, 97, 98, and 99 w-%, and also including equal to about 100 w-% of Compound A, based on the weight of the compound. The remaining material comprises other form(s) of the compound, and/or reaction impurities and/or processing impurities arising from its preparation. For example, a crystalline form of Compound A may be deemed substantially pure in that it has a purity greater than 90 w-%, as measured by means that are at this time known and generally accepted in the art, where the remaining less than 10 w-% of material comprises other form(s) of Compound A and/or reaction impurities and/or processing impurities.

The term “pharmaceutically acceptable excipient” as used herein refers to substances, which do not show a significant pharmacological activity at the given dose and that are added to a pharmaceutical composition in addition to the active pharmaceutical ingredient. Excipients may take the function of vehicle, diluent, release agent, disintegrating agent, dissolution modifying agent, absorption enhancer, stabilizer or a manufacturing aid among others. Excipients may include fillers (diluents), binders, disintegrants, lubricants and glidants.

### Biological Assays, Data and Synthesis

The activity of Compound A according to the present invention has been assessed using the in vitro & in vivo methods described in patent application publication WO2022/249060. Activity data and synthetic routes are provided therein, for the compound of Examples 42 and 123 (Compound A).

1. A crystalline form of Compound A (Modification B), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 5.6±0.2°, 11.2±0.2°, 12.6±0.2°, 14.8±0.2°, 17.4±0.2°, 18.1±0.2°, 19.2±0.2°, 22.0±0.2°, 22.4±0.2°, 25.1±0.2°, 25.3±0.2°, 26.2±0.2°, 27.3±0.2°, 29.7±0.2° and 34.5±0.2°, when measured at a temperature in the range of from 20 to 30° C. with Cu-Kalpha radiation having a wavelength of 0.15418 nm.

2. A crystalline form of Compound A (Modification B), wherein said crystalline form is characterized by a X-ray diffraction pattern substantially the same as the X-ray powder diffraction pattern shown in FIG. 2, when measured at a temperature in the range of from 20 to 30° C. with Cu-Kalpha radiation having a wavelength of 0.15418 nm.

3. A crystalline form of Compound A (Modification C), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 4.0±0.2°, 8.0±0.2°, 8.7±0.2°, 12.1±0.2°, 14.5±0.2°, 15.1±0.2°, 15.5±0.2°, 16.2±0.2°, 16.7±0.2°, 17.9±0.2°, 19.8±0.2°, 20.2±0.2°, 20.6±0.2°, 23.4±0.2°, 24.2±0.2°, 25.4±0.2°, 26.5±0.2°, 28.5±0.2°, 32.1±0.2°, when measured at a temperature in the range of from 20 to 30° C. with Cu-Kalpha radiation having a wavelength of 0.15418 nm.

4. A crystalline form of Compound A (Modification C), wherein said crystalline form is characterized by a X-ray diffraction pattern substantially the same as the X-ray powder diffraction pattern shown in FIG. 3, when measured at a temperature in the range of from 20 to 30° C. with Cu-Kalpha radiation having a wavelength of 0.15418 nm.

5. A crystalline form of Compound A (Modification D), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 8.5±0.2°, 9.1±0.2°, 11.6±0.2°, 13.7±0.2°, 14.3±0.2°, 15.0±0.2°, 15.4±0.2°, 16.9±0.2°, 17.5±0.2°, 19.1±0.2°, 19.9±0.2°, 20.2±0.2°, 21.5±0.2°, 22.6±0.2°, 23.5±0.2°, 25.6±0.2°, 26.4±0.2°, 26.8±0.2°, 28.8±0.2°, 30.3±0.2°, when measured at a temperature in the range of from 20 to 30° C. with Cu-Kalpha radiation having a wavelength of 0.15418 nm.

6. A crystalline form of Compound A (Modification D), wherein said crystalline form is characterized by a X-ray diffraction pattern substantially the same as the X-ray powder diffraction pattern shown in FIG. 4, when measured at a temperature in the range of from 20 to 30° C. with Cu-Kalpha radiation having a wavelength of 0.15418 nm.

7. A crystalline form of Compound A (Hydrate H_{A}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 7.8±0.2°, 8.4±0.2°, 11.8±0.2°, 15.7±0.2°, 16.4±0.2°, 16.8±0.2°, 17.4±0.2°, 17.7±0.2°, 19.7±0.2°, 20.2±0.2°, 24.3±0.2°, 24.7±0.2°, 25.1±0.2°, 26.6±0.2°, 29.0±0.2°, when measured at a temperature in the range of from 20 to 30° C. with Cu-Kalpha radiation having a wavelength of 0.15418 nm.

8. A crystalline form of Compound A (Hydrate H_{A}), wherein said crystalline form is characterized by a X-ray diffraction pattern substantially the same as the X-ray powder diffraction pattern shown in FIG. 5 or FIG. 10, when measured at a temperature in the range of from 20 to 30° C. with Cu-Kalpha radiation having a wavelength of 0.15418 nm.

9. A crystalline form of Compound A (Hydrate H_{B}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 9.0±0.2°, 13.6±0.2°, 14.7±0.2°, 15.8±0.2°, 16.1±0.2°, 16.4±0.2°, 17.4±0.2°, 18.4±0.2°, 19.3±0.2°, 20.3±0.2°, 22.1±0.2°, 22.7±0.2°, 23.1±0.2°, 24.8±0.2°, 25.4±0.2°, 29.6±0.2°, 30.1±0.2°, when measured at a temperature in the range of from 20 to 30° C. with Cu-Kalpha radiation having a wavelength of 0.15418 nm.

10. A crystalline form of Compound A (Hydrate H_{B}), wherein said crystalline form is characterized by a X-ray diffraction pattern substantially the same as the X-ray powder diffraction pattern shown in FIG. 6, when measured at a temperature in the range of from 20 to 30° C. with Cu-Kalpha radiation having a wavelength of 0.15418 nm.

11. A crystalline form of Compound A (Hydrate H_{C}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 8.0±0.2°, 8.7±0.2°, 11.8±0.2°, 12.2±0.2°, 14.9±0.2°, 15.2±0.2°, 15.6±0.2°, 16.4±0.2°, 16.8±0.2°, 19.7±0.2°, 20.1±0.2°, 20.5±0.2°, 21.1±0.2°, 24.3±0.2°, 24.5±0.2°, 25.1±0.2°, 25.6±0.2°, 26.6±0.2°, 33.9±0.2°, when measured at a temperature in the range of from 20 to 30° C. with Cu-Kalpha radiation having a wavelength of 0.15418 nm.

12. A crystalline form of Compound A (Hydrate H_{C}), wherein said crystalline form is characterized by a X-ray diffraction pattern substantially the same as the X-ray powder diffraction pattern shown in FIG. 7, when measured at a temperature in the range of from 20 to 30° C. with Cu-Kalpha radiation having a wavelength of 0.15418 nm.

13. A crystalline form of Compound A (Solvate S_{A}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 7.8±0.2°, 11.8±0.2°, 15.3±0.2°, 15.7±0.2°, 16.8±0.2°, 19.6±0.2°, 20.8±0.2°, 21.9±0.2°, 23.6±0.2°, 24.3±0.2°, 27.6±0.2°, when measured at a temperature in the range of from 20 to 30° C. with Cu-Kalpha radiation having a wavelength of 0.15418 nm.

14. A crystalline form of Compound A (Solvate S_{A}), wherein said crystalline form is characterized by a X-ray diffraction pattern substantially the same as the X-ray powder diffraction pattern shown in FIG. 8, when measured at a temperature in the range of from 20 to 30° C. with Cu-Kalpha radiation having a wavelength of 0.15418 nm.

15. A crystalline form of Compound A (Solvate S_{B}), wherein the crystalline form is characterized by a X-ray powder diffraction pattern comprising 4 or more reflections, preferably 5 or more reflections, more preferably 6 or more reflections, even more preferably 7 or more reflections, at 2-Theta angles selected from a group consisting of: 9.5±0.2°, 11.3±0.2°, 14.4±0.2°, 14.8±0.2°, 15.2±0.2°, 16.9±0.2°, 17.1±0.2°, 17.7±0.2°, 18.0±0.2°, 18.5±0.2°, 18.7±0.2°, 19.1±0.2°, 19.6±0.2°, 20.3±0.2°, 20.9±0.2°, 22.1±0.2°, 23.4±0.2°, 27.1±0.2°, 28.4±0.2°, 28.8±0.2°, when measured at a temperature in the range of from 20 to 30° C. with Cu-Kalpha radiation having a wavelength of 0.15418 nm.

16. A crystalline form of Compound A (Solvate S_{B}), wherein said crystalline form is characterized by a X-ray diffraction pattern substantially the same as the X-ray powder diffraction pattern spectrum shown in FIG. 9, when measured at a temperature in the range of from 20 to 30° C. with Cu-Kalpha radiation having a wavelength of 0.15418 nm.

17. A crystalline form as defined in any one of claims 1 to 16, for the preparation of a pharmaceutical composition.

18. A crystalline form as defined in any one of claims 1 to 16, for use as a medicament.

19. A crystalline form as defined in any one of claims 1 to 16, for use as defined in claim 18, wherein said medicament is for the treatment of cancer, in particular cancer that is characterized as microsatellite instability-high (MSI-H) or mismatch repair deficient (dMMR).

20. A pharmaceutical composition comprising:
a solid form of Compound A as defined in any of claims 1 to 16, or
the composition comprising a solid form of Compound A as defined in any of any of claims 1 to 16,
and at least one pharmaceutically acceptable excipient.

21. A method of treating and/or preventing cancer, in particular cancer that is characterized as microsatellite instability-high (MSI-H) or mismatch repair deficient (dMMR), said method comprising administering an effective amount of the solid form as defined in any of claims 1 to 16, to a patient in need of such a treatment.

22. A process for the preparation of the crystalline form as defined in any one of claims 1 to 16.


SEQUENCE LISTING (2 sequences)

SEQ ID NO: 1 - DNA, 9674 bp, synthetic construct
  misc_feature 1..9674: note = Description of Artificial Sequence: Syntheticpolynucleotide
  source 1..9674: mol_type = other DNA
aaccatctcgcaaataaataagtattttactgttttcgtaacagttttgtaataaaaaaacctataaatattccggattattcataccgtcccaccatcgggcgccatggcttctcaccaccatcaccatcaccatcatcatcacgctcagcacgacgaggctgtggacaacaagttcaacaaggagcagcagaacgctttctacgagatcctgcacctccctaacctgaacgaggagcagcgtaacgctttcatccagtccctgaaggacgacccttctcagtctgctaacctgctggctgaggctaagaagctgaacgacgctcaggctcctaaggtggacaacaagttcaacaaggagcagcagaacgctttctacgagatcctgcacctccctaacctgaacgaggagcagcgtaacgctttcatccagtccctgaaggacgacccttctcagtctgctaacctgctggctgaggctaagaagctgaacgacgctcaggctcctaaggtggacgctaacggtggcggcggttccggcggtggtggctctctcgaggttctgttccagggtccgaatgaaggcgaggaagatgatgacaaagacttcctgtggccagctccaaacgaagaacaggtgacttgtctcaagatgtacttcggtcatagcagcttcaaaccagtgcagtggaaagttatccacagcgttcttgaagaacgtcgtgataatgtggctgtgatggctactggctatggtaagagcctgtgtttccagtacccgccagtttacgttggtaagatcggtctggtgattagcccgctgatctctctgatggaagaccaggtgctgcaacttaagatgagcaacatcccggcttgtttcctgggttctgcacaaagcgagaacgtgctcaccgatatcaagctgggtaagtaccgtatcgtgtacgtgacgccagaatactgtagcggcaacatgggtcttctgcaacagctcgaagctgatattggcatcaccctcattgcagtggacgaagctcactgtatcagcgagtggggtcatgatttccgcgactctttccgtaaactgggttctctgaagactgcacttccgatggttccaattgtggcactgaccgcaactgcttctagctctattcgtgaagacatcgttcgttgcctgaacctccgtaacccacaaattacctgcaccggctttgaccgtccgaacctgtacctggaggttcgtcgtaagaccggtaatatccttcaggacctgcaaccattcctggttaagaccagcagccactgggagttcgaaggtccgactatcatctactgcccaagccgtaagatgacccagcaggttactggtgaactgcgtaaactgaacctgagctgtggcacttaccacgcaggcatgtctttctctacccgtaaagacatccatcatcgtttcgtgcgtgatgaaatccagtgcgttatcgctaccattgcattcggcatgggtatcaacaaagctgacatccgtcaagtgattcactacggtgcaccgaaagacatggaaagctactaccaggaaatcggccgtgcaggtcgtgatggtctgcaaagctcttgtcatgtgctgtgggcaccagcagatattaacctgaaccgtcacctgctgactgaaattcgtaacgagaaattccgtctgtacaaactgaagatgatggcgaagatggagaaatacctgcatagctcccgttgtcgtcgtcaaatcattctgagccatttcgaggataaacaggtgcagaaagcttctctgggtatcatgggcactgagaagtgctgcgataactgtcgtagccgtcttgatcactgctacagcatggacgatagcgaagacacttcttgggatttcggtccacaagcattcaaactgctgagcgcagttgatatcctgggtgagaaattcggcatcggcctcccaatcctgtttctgcgcggttctaactctcagcgtcttgctgatcaataccgtcgtcactctctgttcggcactggtaaagaccagaccgaatcttggtggaaagcattcagccgtcaacttatcaccgaaggctttctggtggaagtgtctcgttacaacaagttcatgaagatctgcgcactgactaagaaaggtcgtaactggctgcacaaggcaaataccgagtctcagtctcttatccttcaggctaacgaagaactgtgcccgaagaagcttctgctgccatcttctaagaccgtgagctctggtactaaagagcattgctacaaccaggtgccggttgaactgtctaccgagaagaagtccaacctggagaagctgtactcctacaaaccgtgcgacaagatctcctccggttctaatatcagcaagaagtccatcatggtgcagtctccggagaaagcttacagcagctctcagccagttatctctgcacaggaacaggaaactcagattgtgctgtacggtaaactggtggaagcacgtcagaaacacgctaacaagatggacgtgccgccagcaattcttgcaaccaacaagattctggtggacatggctaagatgcgcccaactactgttgagaacgtgaaacgtatcgacggtgttagcgaaggtaaagctgcaatgctggcaccactgcttgaagttatcaagcatttctgccagaccaactctgttcagaccgacctgttctcttctaccaaaccataatggtaccgaattcgcggccgcagagctcgctctggtgccacgcggtagttccgcttggagccacccgcagttcgaaaagtaagtgattaacctcaggttatacatatattttgaatttaattaattatacatatattttatattatttttgtcttttattatcgaggggccgttgttggtgtggggttttgcatagaaataacaatgggagttggcgacgttgctgcgccaacaccacctcccttccctcctttcatcatgtatctgtagataaaataaaatattaaacctaaaaacaagaccgcgcctatcaacaaaatgataggcattaacttgccgctgacgctgtcactaacgttggacgatttgccgactaaaccttcatcgcccagtaaccaatctaggtagctgagcgcatgcaagctgatccgggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctaagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgaggtttaaacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccaggaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaataagacgcacaaactaatatcacaaactggaaatgtctatcaatatatagttgctgatcagatctacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatcgtcgagatcccgggcgtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaacttgcaattggacacggcatagttttccatagtcgtgtaaaacatggtattggctgcattgtaatacatccgactgagcgggtacggatctatgtgtttgagcagcctgttcaaaaactctgcatcgtcgcaaaacggaatttggtacccgggcgtatactccggaatattaatagatcatggagataattaaaatgat

SEQ ID NO: 2 - DNA, 45 bp, synthetic construct
  misc_feature 1..45: note = Description of Artificial Sequence: Syntheticoligonucleotide
  source 1..45: mol_type = other DNA
ttttttttttttttttttttttccaagtaaaacgacggccagtgc